(19) United States
(12) Patent Application Publication
(10) Pub. No.: US 2020/0000963 A1
(43) Pub. Date: Jan. 2, 2020

(54) AUTOMATED PROCESS FOR THE PRODUCTION OF HUMAN OR ANIMAL TISSUES FOR GRAFTS

(71) Applicant: THERACELL CONSULTING SPRL, Lasne (BE)

(72) Inventor: Denis Dufrane, Lasne (BE)

(21) Appl. No.: 16/473,497

(22) PCT Filed: Dec. 19, 2017
(86) PCT No.: PCT/EP2017/083540

§ 371 Date: Jun. 25, 2019

(30) Foreign Application Priority Data
Dec. 26, 2016 (BE) BE2016/5978

(51) Int. Cl. A61L 27/36

(52) U.S. Cl. CPC A61L 27/3691; A61L 2430/40; A61L 27/3604

(57) ABSTRACT

The present invention proposes a process for obtaining a tissue matrix for allograft or xenograft, according to which, after recovery of a biological tissue, said tissue is classified, said tissue is treated, and the tissue matrix obtained is packaged, all these steps being carried out in an automated way within a same “classified” reactor. The invention also proposes the reactor to implement the process of the invention.

The present application concerns the field of the production of human or animal tissues for use in grafts (allografts or xenografts).

An allograft involves a human donor and recipient. An autograft involves only one human being.

A xenograft involves an animal donor and a human recipient.

A growing number of human musculoskeletal disorders require treatment by bone reconstruction or substitution, both in dentistry (maxillofacial reconstruction by dental implant), oncology (following the removal of bone tumors) or orthopedic surgery (following a fracture or for vertebrae fusion).

Currently, bone reconstruction is done by autograft, usually by puncturing bone from the iliac crest of the person requiring treatment. However, in addition to being painful, this puncture is not reversible. In effect, the iliac bone does not regenerate, and the amount of bone tissue available is thus limited. Autografting requires, moreover, a double surgical intervention on the same individual, which also has a significant impact on post-operative recovery.

Another approach is the use of widely accessible and inexpensive bone substitutes, such as ceramic, coral or glass prostheses.

Unfortunately, due to the weak regulation in this market, many problems of reliability, material compatibility and even toxicity are at the root of many post-operative problems, and medical personnel are increasingly suspicious vis-a-vis these substitutes.

Allografting is also practiced. In practice, bone tissue is removed from an individual, for example, a femoral head removed during hip replacement surgery. This tissue contains the bone matrix as well as donor cells. In order to eliminate the risk of rejection, or inflammatory response, during transplantation, it is necessary to treat the tissue in order to clean it of any “genetic” traces from the donor, i.e. to eliminate traces of cells, blood or fat.

The method of cleaning or “decellularization” of the bone tissue to provide a decellularized bone matrix is described in Dufrane et al., Biomaterials. 2002 July; 23(14):2979-88 and Dufrane et al. Eur Cell Mater. 2001 Jan. 10; 1:52-8; discussion 58.

The main steps of this method are:
Centrifugation of tissue taken from the donor to remove blood and fat;
Cutting the tissue, generally into cubes of varying sizes;
Chemical treatment, in order to eliminate traces of cells, and to inactivate viruses and/or bacteria;
Lyophilization, to obtain stable, demineralized bone matrix,
Packaging.

Although allografting is the most reliable bone reconstruction technique to date, patients' access to this technique is severely restricted by the method and costs of producing the decellularized bone matrix.

In effect, at present, all these steps are carried out manually, by hospital staff, in a so-called “classified” sterile clean room, i.e. built according to a certain number of standards and subject to strict controls.

These steps must first be carried out each in different classified sterile enclosures, or isolators, within the production area, each isolator containing the appropriate equipment for each step, and must be kept sterile. The result of this is a large dead space for staff to move between each isolator. It is also necessary to take special precautions when transferring production batches from one isolator to another.

It is then necessary to ensure the traceability of the batches as to the origin of the tissue. Mistakes in handling by personnel cannot be ruled out and may lead to the exclusion of batches.

Furthermore, the financial aspect is also significant. In effect, the production of a batch takes several weeks, this duration possibly being influenced by the availability of hospital staff who must carry out their main care activities. The demand for working time, as well as for highly regulated dedicated space, represents a significant cost for a hospital facility. For these reasons, many hospital facilities do not invest in the production of decellularized bone matrices. Access to this bone source for allograft is therefore currently very limited.

In summary, the problems encountered are related to the fact that:
production is done manually, which implies:
   a relative classification of the different steps,
   a need for significant workspace,
   handling and
   traceability difficulties;
manipulation by operators may lead to errors and affect product quality and
the duration of the production generates prohibitive costs. “Relative classification” is intended here to mean keeping the biological tissue under sterile conditions, not only during each step, but also within each step, in the event of a possible physical movement of the tissue between two sterile areas.

These production problems are not limited to bone tissue but are also encountered for other biological tissues used in allografting, such as skin, tendons, bladder, etc., or adipose tissue. These same problems are also encountered in the production of cellular therapies.

In order to improve patients' access to this reliable source of biological tissue for allografts, the applicant has perfected a process to solve the aforementioned problems.

### Solution of the Invention

To this end, the present invention proposes a process for obtaining a tissue matrix for allograft or xenograft, according to which, after recovery of a biological tissue:
said tissue is classified,
said tissue is treated and
the tissue matrix obtained is packaged,

characterized in that these steps are carried out in an automated way within a same “classified” reactor.

In order to solve all the problems, the applicant boldly selected one of them, and namely the one which was a priori the most difficult to solve, but realized that by seeking to eliminate as much the classification as much as possible, she was freed from all the other difficulties. To this end, the present application concerns a problem invention.

“Classifying” the tissue is intended to mean preparing it for introduction into a sterile environment that meets the standards required for the process. Similarly, the “classified” reactor is a reactor that meets these same standards.

“Packaging” refers to any form of packaging, such as for example the introduction of the tissue matrix into a vacuum-sealed plastic bag, or the introduction of the tissue matrix into a ready-to-use syringe, which is itself packaged in plastic in a sterile way.

For some biological tissues, the process of the invention may comprise a step of cutting or grinding the tissue. It may furthermore comprise a drying or lyophilization step prior to packaging.

The treatment of the tissue may comprise one or more of the following steps:
centrifugation of the tissue;
chemical treatment;
biological treatment.

A chemical treatment comprises any exposure of the biological tissue to chemical agents or reagents, such as agitating the tissue in a solution containing an antibacterial agent or a solution in which the pH has been adjusted acid or basic, by adding ionic compounds. The biological tissue may undergo several consecutive chemical treatments with different purposes, such as, for example, fat removal or inactivation of bacteria or viruses or prion or demineralization to increase osteoconduction.

A biological treatment refers to the application of biological agents to the tissue concerned, such as, for example, growth factors or proteins inducing a cellular differentiation.

The invention also concerns the classified reactor for the implementation of the process of the invention, which comprises
an airlock for the introduction and classification of a biological tissue;
biological tissue treatment means arranged to produce a tissue matrix;
packaging means for the tissue matrix produced;
an exit airlock for the packaged tissue matrix;
robotic means for moving biological tissue from the introduction and classification airlock to the treatment means of the biological tissue, and
robotic means for moving the tissue matrix from the treatment means to the packaging means, then from the packaging means to the exit airlock.

The airlock for introducing and classifying a biological tissue, as well as the exit airlock of the packaged tissue matrix, are arranged to maintain sterile conditions within the reactor that meet the standards required for the process.

The tissue is thus classified only at the level of the introduction airlock. The exit airlock is of course also envisaged so that sterile conditions are maintained when the product leaves. The classified reactor may thus be arranged in a compact way, not necessarily allowing an individual to penetrate therein. All the steps of the process of the invention may thus be implemented according to an optimized sequence.

The invention will be better understood with the help of the following description of several embodiments of the invention, with reference to the accompanying drawing wherein:

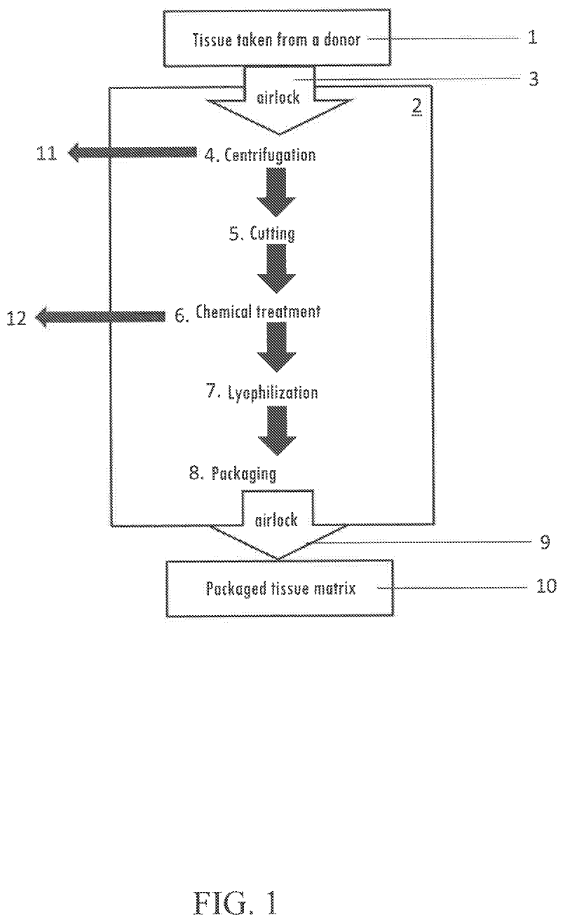

FIG. 1 is a schematic illustration of the process of the invention;

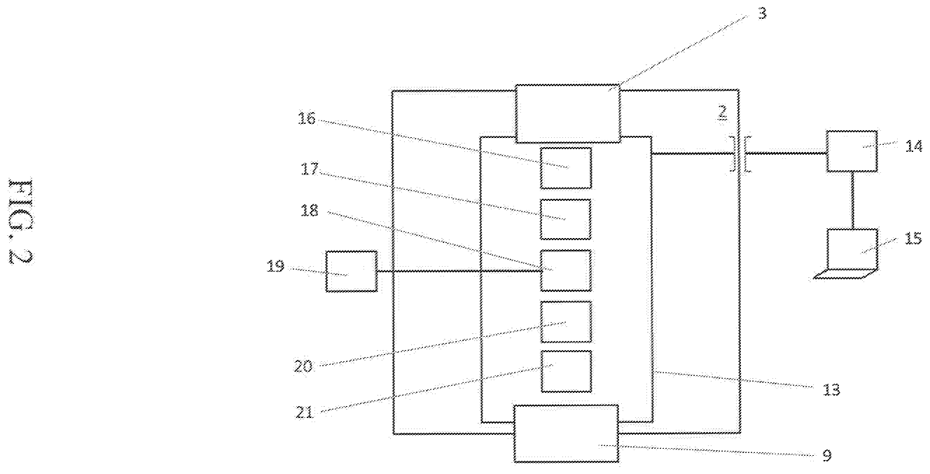

FIG. 2 is a schematic illustration of a reactor for the implementation of the invention, and

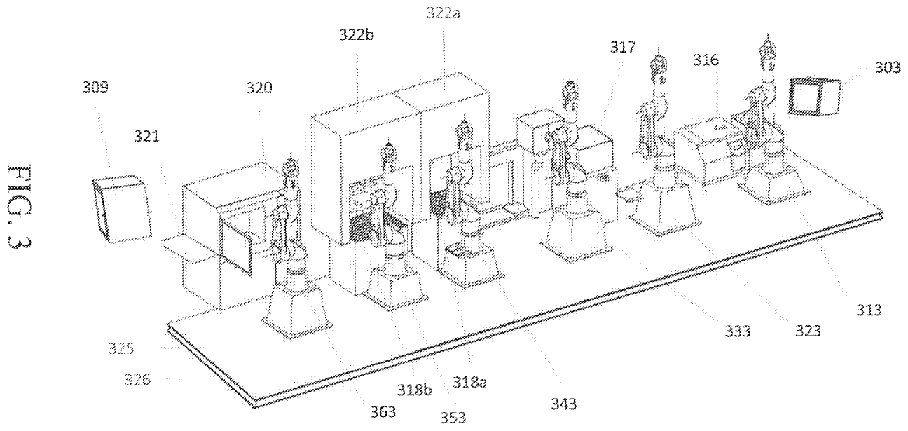

FIG. 3 shows, in perspective, the inside of a reactor according to the invention.

With reference to FIG. 1, a biological tissue 1, taken from a donor, is introduced into a reactor 2 via an entry airlock 3. The tissue undergoes a number of steps: centrifugation 4, cutting 5, chemical treatment 6, lyophilization 7 and packaging 8. The resulting packaged tissue matrix 10 then exits from the reactor 2 via an exit airlock 9.

The tissue 1 may, for example, be a femoral head, taken from an individual donor, when a hip prosthesis is put in place. This raw femoral head, in order to be used to manufacture bone matrix for its use in a bone reconstruction in another patient, must undergo a certain number of transformations in a controlled sterile environment. The femoral head will therefore first be introduced into the so-called “classified” reactor 2, in the sense that it has a sterile environment corresponding to the standards required for these transformations.

The femoral head 1 is introduced via the entry airlock 3, which is configured in such a way that no contamination from outside may penetrate in the reactor 2. This airlock 3 may, for example, be equipped with a double door system, each door opening alternately, and a conventional laminar air flow, directed towards the outside of the airlock. When introduced, it is conceivable to place the femoral head in a special container that will then facilitate its use during the steps of the process. The airlock 3 thus makes it possible to introduce and classify the biological tissue, in this case the femoral head 1, entering the reactor 2.

With reference to FIG. 2, inside the reactor 2, a robot 13, or a series of robots cooperating with each other, comprising units for various functions, is arranged to allow the implementation of the steps of the process. This robot 13 is advantageously controlled by a computer system 14 to which instructions may have been given by an operator by means of a control station 15, in this case a computer.

The reactor may take various shapes and sizes. For example, it may be made of metal, such as a road or sea transport container, or have a part of its walls glazed. The only limitations on the appearance of this reactor are related to the constraint of it being able to establish and maintain a sterile environment that meets the standards required for the manufacturing process. These constraints and standards may vary according to geographical areas. A number of elements may be integrated into this reactor to ensure a sterile environment, such as, for example, ventilation, decontamination or air conditioning units.

Here, the robot 13 introduces the femoral head 1, possibly with its container, into a centrifugation unit 16 where it is centrifuged, in order to separate the bone matrix from unwanted residues 11, such as marrow, blood or fat residues. The femoral head 1, thus cleaned, is then transferred to a cutting unit 17, configured to produce bone pieces of a predefined size. These pieces are then introduced into an enclosure containing a chemical treatment bath 18 where they will be agitated for a predefined period of time. The treatment bath may, for example, be a disinfectant solution to inactivate the bacteria or any other bath adapted to the nature of the treated tissue.

The enclosure 18 wherein the chemical treatment is carried out may be arranged in such a way that, at the end of the treatment, the chemical solution used 12 may be discharged to a suitable container 19 attached to the reactor 2. It is conceivable, for example, that this container is located outside and isolated from the reactor through the use of valves, so that it may be replaced when it is full, without affecting the sterile environment in the reactor. It is conceivable to eliminate the residues from centrifugation in the same way. It is also conceivable to connect containers of “clean” solutions that will be used to supply the chemical baths. The content of these containers is easily traceable by the computer system 14, for example via a barcode system.

Several subsequent chemical treatments may occur on the pieces of bone, within the same treatment enclosure, or in separate enclosures inside the reactor 2. They may be completed by rinsing steps. The decellularized bone matrix pieces thus treated are then lyophilized in a lyophilizer 20, i.e. dried until they contain, for example, less than 5% moisture, and then packed in a vacuum or in a protective atmosphere in a packaging unit 21. This ensures an optimal stability of the bone matrix 10, allowing it to be stored at room temperature for several months. It is conceivable to include a package labeling step to ensure the traceability of the batches produced. The packaged tissue matrix 10 is extracted from the reactor 2 through the exit airlock 9, similar to the entry airlock 3. It is also possible to configure the reactor 2 to have only one entry and exit airlock.

The command and control station 15 comprises the computer system 14 into which is entered the manufacturing process of the packaged tissue 10. The computer system 14 controls the robot 13 in the reactor 2. Some parameters may need to be entered by the operator, such as the nature of the tissue, whether the robot may manage the production of several types of tissues, the duration of chemical treatments or the desired percentage of moisture in the packaged tissue matrix 10.

The manufacturing process described above thus makes it possible to introduce a “raw” biological tissue into a reactor and to recover at the exit a decontaminated biological tissue ready to be used for a graft. There is only one classification step in this process, at the entry of the tissue 1 into the reactor 2. The continuity of the classification is ensured by the restricted and controlled space of the reactor from which the tissue does not leave before the end of production. As no operator need intervene during the process, manipulation errors are excluded, traceability is ensured, space and handling needs are reduced strictly to a minimum. As a result, production costs are also optimized.

Another advantage of the process of the invention is the possibility of managing “small” productions continuously. Usually, when the production is carried out manually in a hospital complex (via a tissue bank), hospital staff wait until they have a sufficient quantity of starting material, several dozen femoral heads, for example 48, before starting a production in order to optimize costs. The process of the invention makes it possible to start a production with a single femoral head and ensure continuous production, or at each time that a new femoral head is available.

The reactor 2 may be advantageously arranged to allow the simultaneous production, in parallel or staggered, of several batches of tissue matrix. That is to say, two raw biological tissues may be introduced and processed at the same time, or a second biological tissue may be introduced while a first tissue is already being processed. The traceability of tissue matrix batches is ensured for each biological tissue.

It is apparent to the person skilled in the art that the tissue 1 may also be collected from an animal donor. The treatment of femoral heads has been described here, but it could also be the treatment of any other bone, skin or other tissue conceivable by the person skilled in the art.

The process of the invention is not limited to the production of tissue matrices. Cellular therapies, or biotherapies, may also benefit from the process of the invention. These therapies are a form of autograft frequently used in oncology and where specific differentiated cells are produced from stem cells taken from an individual for reimplantation in the organ to be treated. The preparation of these therapies, which until the present time has been done manually, suffers from the same problems as the production of tissue matrices. Their manufacture also comprises classification, treatment and packaging steps.

Ideally, the reactor 2 is easily transportable by any means of transport and may be easily installed anywhere in the world. The process of the invention does not require the intervention of highly qualified hospital personnel and may be adapted to the standards in force where it is implemented.

A particular embodiment of the reactor of the invention is detailed in FIG. 3, representing the internal arrangement of a reactor of the invention. The walls delimiting the reactor are not represented, for reasons of clarity, but it is quite obvious that they surround, in the manner of a sealed container, the equipment contained in the reactor. “Sealed” is intended to mean that the reactor does not comprise any openings, other than the airlocks and openings specifically designed for technical needs.

The reactor 302 comprises an airlock 303 for the introduction and classification of biological tissues taken from a donor and an airlock 309 for the exit of a packaged tissue matrix, each airlock being placed at an end of the reactor. Vials 327, containing biological tissues, are shown here in the airlock 303. Between the two airlocks are placed, side by side, five pieces of equipment: a centrifuge 316, a cutting unit 317, two chemical treatment stations 318a and 318b, each placed under a fume hood 322a and 322b, a lyophilizer 320 and a packaging station 321, in front of which are positioned five articulated robotic arms 313, 323, 343, 353 and 363 respectively, as well as an articulated robotic arm 333 positioned between the cutting unit 317 and the chemical treatment station 318a.

All the elements described above are placed on a floor 325 under which is provided a space 326.

The elements of the reactor 302 having been described, the articulation of the elements between them for the implementation of the process of the invention will now be detailed.

The airlock 303 for the introduction and classification of biological tissues is similar to the airlock 3 in FIGS. 1 and 2. After opening the outer door of the airlock, an operator places in the airlock 303 a biological tissue to be treated, in a vial 327. The air flow inside the airlock is such that no outer contamination may penetrate the airlock until the outer door is closed again.

The automated treatment process begins with the opening of the inner door (to the reactor) of the airlock 303. The robotic arm 313 grips the vial 327 in the airlock 303 and deposits it in a cavity of the centrifuge 316. The vial 327 may, for example, be arranged to match the working cavities of the centrifuge. It may have a double wall, the inner wall being perforated, so that substances extracted from the biological tissue during centrifugation pass through the perforated wall and may thus be physically separated from the biological tissue.

Several batches, in several vials, may possibly be centrifuged simultaneously. During the process, it is easy to follow the position of each batch in all the pieces of equipment without the possibility of error.

After centrifugation, the robotic arm 313 takes the vial 327 out of the centrifuge 316. The arm 323 takes the biological tissue, which has been rid, at least in part, of undesirable elements, in order to proceed with its cutting.

It is also possible to provide an intermediate platform, where the vials containing the centrifuged biological tissue are stored before being taken by the arm 323.

Cutting may be done in the traditional way, using a band saw or a mill. These methods, however, have several disadvantages. First, friction with the biological tissue generates heat, usually above 47° C., which damages this tissue. Particularly when it involves a bone, the cavities of the bone become blocked due to heat and pressure applied on the cutting surface. Secondly, in theory, it is necessary to change the cutting band to avoid contamination between batches, which is rarely done when the process is performed by technicians in a hospital. Finally, these methods, which are geometrically quite inflexible (4-axis machines), generate a considerable loss of biological tissue.

To overcome these disadvantages, the applicant has innovatively integrated into the automated process a cutting using a non-abrasive, high-pressure water jet. This technique has already been used manually by operating room surgeons, as described in the Journal of The Mechanical Behavior of Biomedical Materials, 62, 2016, 495-503, to partially cut bones during an operation. Using a high-pressure water jet, on the one hand, allows one to cut very precisely and cleanly, without elevation in temperature that could damage the tissues, and, on the other hand, allows one to not cross-contaminate the batches.

Another advantage of using a high-pressure water jet is that it may work for any tissue, whether bone or skin. The pressure of the water jet may be adapted to the tissue to be cut. The cut is clean, with a micrometric precision. In the case of bone, the bone cells at the cutting interface are not damaged.

In practice, the arm 323 holding the biological tissue to be cut is movable in several axes simultaneously and positions and moves the tissue to be cut under the jet according to any predefined orientation. Since the articulated arm is movable according to several axes, it is possible to obtain any cutting geometry, which also makes it possible to optimize cutting in order to limit waste and obtain a higher yield of tissue matrix at the end of the process.

The pressurized water jet is generated in equipment well known to the person skilled in the art, comprising a compressor and an outlet nozzle, for example made of sapphire or diamond to limit wear and tear and with an adjustable diameter to adjust the pressure and precision of the cut.

Although high-pressure water jet cutting is technically more efficient, the invention is not limited to this mode of cutting and all conventional cutting methods may be used here.

Once cut, the pieces of tissue are recovered, for example on a screen, before being removed, one by one, by the arm 333, which places them in a basket, batch by batch, in order not to mix pieces from different batches and to ensure complete traceability.

The arm 343 introduces the baskets into the chemical treatment baths, as described above, and then removes them once the treatment is finished. It may also manage intermediate steps or several different successive chemical treatments on the platforms 318a and 318b. The fume hoods 322a and 322b are conventional laboratory fume hoods, which regulate the air quality in the reactor above the chemical solutions. Once the chemical treatment has finished, the robotic arm removes the baskets and places them near the robotic arm 353.

The robotic arm 353 picks up the treated pieces of biological tissue one by one to place them each in a pre-sealed tube, i.e. a tube with caps already arranged but still not sealed, and then introduces each tube into the lyophilizer 320. At the end of lyophilization, before reopening the lyophilizer, a plate is pressed against the caps to seal the tubes, each containing a piece of tissue matrix, ready to be used for a graft.

The arm 363 will then finish packaging the tubes, for example by applying a label including all the information necessary for batch traceability, and then place the packaged tissue matrix tubes in the exit airlock 309 so that they may be retrieved by an operator.

The space 326 provided under the floor 325 may be used to run all the cables and tubes necessary for the electrical, computer and fluidic connection of equipment and for the evacuation of waste generated during the process.

The robotic arms may of course perform other movements than those described above to ensure the proper operation of the entire process.

The layout of the equipment is here described in a line, but any other arrangement allowing the implementation of the process of the invention is conceivable. In particular, it is possible to use fewer robotic arms or even a single robotic arm to perform all the operations among the pieces of equipment, which would be distributed around it.

The robotic means, described above, are illustrated here by one or more robotic arms, fixed to the ground. Nevertheless, any other configuration or equivalent equipment may be conceived, whether suspended, and/or on rail, with gripping capabilities or specifically configured for a precise material. The notion of a robotic arm is to be taken in the very broad sense of an automated, programmable and/or remotely controllable means. A conveyor belt may also be used to assure the movement of tissues, matrix, waste or any other element that needs to be moved.

The equipment described here is conventional equipment such as a centrifuge or lyophilizer. Depending on the tissue treated, other types of equipment may be used, or custom-made equipment may be developed. The means of treatment, cutting and packaging are to be taken in the broadest sense and comprise all the means known to the person skilled in the art.

1. A process for obtaining a tissue matrix for allograft or xenograft, including, after recovery of a biological tissue:
classifying said tissue,
treating said tissue to produce a tissue matrix, and
packaging the tissue matrix;
wherein the steps are carried out in an automated way within a same “classified” reactor.

2. The process according to claim 1, further comprising cutting said tissue.

3. The process according to claim 1, further comprising lyophilizing the tissue matrix prior to said packaging.

4. The process according to claim 1, wherein the treating the tissue comprises at least one of the steps of the group including the steps of:
centrifugating the tissue,
chemically treating the tissue,
biologically treating the tissue.

5. The process according to claim 1, wherein the tissue matrix is a decellularized bone matrix.

6. The process according to claim 1, wherein the tissue matrix is a cellular therapy.

7. The process according to claim 2, wherein the cutting includes cutting the tissue with a high-pressure water jet.

8. A reactor for implementing a process of obtaining a tissue matrix for allograft or xenograft, the reactor comprising:
an airlock for the introduction and classification of a biological tissue;
biological tissue treatment means arranged to produce a tissue matrix;
packaging means for packaging the tissue matrix produced;
an exit airlock for the packaged tissue matrix; and
first robotic means for moving the biological tissue from the introduction airlock to the biological tissue treatment means and second robotic means for moving the tissue matrix from the biological treatment means to the packaging means and then to the exit airlock.

9. The reactor according to claim 8, further comprising means for cutting the biological tissue.

10. The reactor according to claim 9, wherein the cutting means comprises a non-abrasive, high-pressure water jet.

11. The reactor according to claim 8, wherein the first robotic means and/or the second robotic means comprise robotic arms.
